(19) United States
(12) Patent Application Publication
(10) Pub. No.: US 2022/0203049 A1
(43) Pub. Date: Jun. 30, 2022

(54) SYSTEM, INHALER, AND METHOD OF MONITORING

(71) Applicant: KINDEVA DRUG DELIVERY L.P., Woodbury, MN (US)

(72) Inventors: Christopher G. BLATCHFORD, Bracknell (GB); David COOKS, Bracknell (GB)

(73) Assignee: KINDEVA DRUG DELIVERY L.P., Woodbury, MN (US)

(21) Appl. No.: 17/608,303

(22) PCT Filed: Apr. 30, 2020
(86) PCT No.: PCT/IB2020/054079

§ 371 Date: Nov. 2, 2021

Related U.S. Application Data
(60) Provisional application No. 62/844,216, filed on May 7, 2019.

(51) Int. Cl. A61M 15/00

(52) U.S. Cl. CPC A61M 15/0021; A61M 2230/42; A61M 2205/13; A61M 2205/3375

(57) ABSTRACT

A system for use with an inhaler includes an actuator housing. The system includes a loudspeaker disposed within the actuator housing. The loudspeaker is configured to emit an acoustic pulse during use of the inhaler by a patient. The system also includes a microphone disposed within the actuator housing. The microphone is configured to receive reflected sound generated in response to the acoustic pulse. The system further includes a controller communicably coupled to the microphone. The controller is configured to determine a parameter of the oral cavity of the patient based on the reflected sound.

### FIELD

The present disclosure generally relates to inhalation devices and, more particularly, to monitoring the use of inhalation devices.

### BACKGROUND

For various reasons, a certain percentage of patients suffering from chronic illnesses, such as asthma and chronic obstructive pulmonary disease (COPD), do not take their prescription as prescribed. This can inhibit patient improvement and cause disease progression. Hence, adherence programs can be used to measure an extent to which patients follow their prescribed medication for treatment of their health condition.

Inhalers for pulmonary delivery, whether they be press-and-breathe or breath-actuated type devices, can deliver a medicament to an oral cavity of a patient. The medicament is delivered through an orifice which is in fluid communication with a fluid source, such as a canister.

### SUMMARY

Inhalers often include a mouthpiece, a portion of which is received within a mouth of the patient during use of the inhaler. It can be important to position the mouth correctly around the mouthpiece during inhaler use, as improper positioning of the mouth (e.g., lips and tongue) while administering the medicament may lead to poor delivery of the medicament to target areas. This may retard patient improvement and encourage disease progression. By way of example, if the lips are not properly sealed around the mouthpiece, then the desired flow of medicament to the airways may not be achieved. Also, if the tongue is in a high position in the mouth, then it may block the delivery of medicament from the mouthpiece of the inhaler to the airways.

In one aspect, the present disclosure relates to a system for use with an inhaler. The inhaler includes an actuator housing having a mouthpiece. The system includes a loudspeaker disposed within the actuator housing. The loudspeaker is configured to emit an acoustic pulse during use of the inhaler by a patient. The system also includes a microphone disposed within the actuator housing. The microphone is configured to receive reflected sound generated in response to the acoustic pulse. The system further includes a controller communicably coupled to the microphone. The controller is configured to analyze the reflected sound received by the microphone.

In another aspect, the present disclosure relates to a method of monitoring use of an inhaler by a patient. The inhaler includes an actuator housing. The method includes emitting, within the actuator housing, an acoustic pulse during use of the inhaler. The method also includes receiving, within the actuator housing, reflected sound generated in response to the acoustic pulse and analyzing the reflected sound. The method further includes determining a parameter of the oral cavity of the patient based on the reflected sound.

In another aspect, the present disclosure relates to an inhaler for delivering a medicament to a patient. The inhaler includes an actuator housing comprising a mouthpiece. The inhaler also includes a loudspeaker disposed within the actuator housing. The loudspeaker is configured to emit an acoustic pulse during use of the inhaler by the patient. The inhaler further includes a microphone disposed within the actuator housing. The microphone is configured to receive reflected sound generated in response to the acoustic pulse. The inhaler includes a controller communicably coupled to the microphone. The controller is configured to analyze the reflected sound received by the microphone.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a perspective view of an inhaler according to embodiments of the present disclosure;

FIG. 2 illustrates exemplary usage of the inhaler of FIG. 1 by a patient;

FIG. 3 is a block diagram of a system for use with the inhaler depicted in FIG. 1 according to embodiments of the present disclosure;

FIG. 4 illustrates an exemplary plot for an acoustic pulse;

FIG. 5 is an exemplary plot illustrating comparison between a detected signal pattern and predetermined signal patterns to detect tongue positioning during use of the inhaler of FIG. 1;

FIG. 6 is another exemplary plot illustrating comparison between a detected signal pattern and predetermined signal patterns to detect tongue positioning during use of the inhaler of FIG. 1;

FIG. 7 is an exemplary plot illustrating comparison between a detected signal pattern and predetermined patterns to detect sealing of lips with the mouthpiece during use of the inhaler of FIG. 1;

FIG. 8 is another exemplary plot illustrating comparison between a detected signal pattern and predetermined signal patterns to detect sealing of lips with the mouthpiece during use of the inhaler of FIG. 1; and

FIG. 9 is a flowchart for a method of monitoring use of the inhaler by the patient according to embodiments of the present disclosure.

### DETAILED DESCRIPTION

In the following description, reference is made to the accompanying figures that form a part thereof and in which various embodiments are depicted by way of illustration. It is to be understood that other embodiments are contemplated and may be made without departing from the scope or spirit of the present disclosure. The following detailed description, therefore, is not to be taken in a limiting sense.

The present disclosure will be described with respect to particular embodiments and with reference to certain drawings, but the disclosure is not limited thereto. The drawings described are only schematic and are non-limiting. In the drawings, the size of some of the elements may for illustrative purposes be exaggerated and not drawn to scale.

It will be understood that the terms “vertical”, “horizontal”, “top”, “bottom”, “above”, “below”, “left”, “right” etc. as used herein refer to particular orientations of the figures and these terms are not limitations to the specific embodiments described herein.

Inhalers can comprise a canister-retaining tubular housing portion and a tubular mouthpiece portion, the tubular mouthpiece portion can be angled with respect to the tubular housing portion. An air inlet is defined at an upper end or a lower end of the tubular housing portion. Proximal to the lower end of the tubular housing portion, a thumb grip is provided. Further, a metering valve is disposed within the tubular housing portion that releases a metered amount of medicament from a canister or reservoir of the inhaler During operation of the inhaler, a plume of medicament produced from an orifice that is in communication with the metering valve is introduced into the tubular mouthpiece portion and is inhaled by a patient through the tubular mouthpiece portion. However, as described above, patients do not necessarily always position their mouth correctly around the mouthpiece during inhaler use.

Therefore, it can be helpful to interrogate the patient's mouth shape, in particular a position of their tongue, and whether the patient has their lips sealed around the mouthpiece and accordingly notify the patients when the mouth is not positioned properly as the patient may not receive the full dose due to such improper positioning.

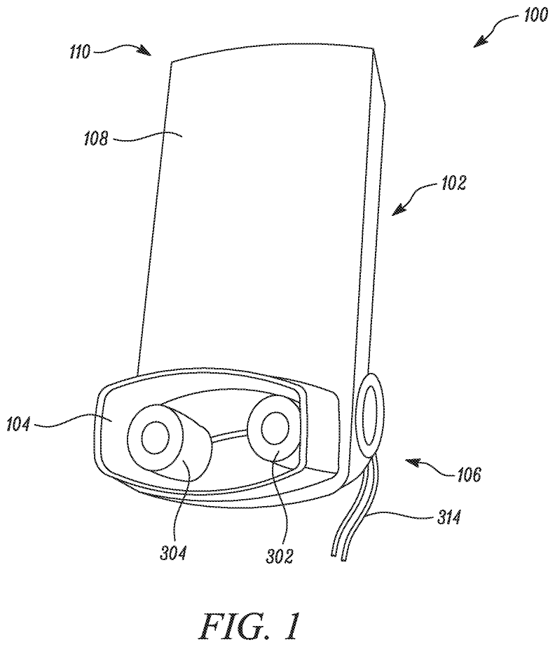

FIG. 1 illustrates a perspective view of an inhaler 100 for delivering a medicament to a patient. In some embodiments, the inhaler 100 may embody an electronic inhaler. The inhaler 100 may include an onboard power source (not depicted), such as batteries or cells, that powers various electronic components of the inhaler 100. Further, the inhaler 100 may be, by way of example, a press-and-breathe inhaler or a breath-actuated inhaler. The press-and-breathe inhaler or the breath-actuated inhaler may comprise a Pressurized Metered-Dose Inhaler (pMDI). In another example, the inhaler may comprise a Dry Powder Inhaler (DPI). In embodiments, the inhaler may comprise a soft-mist inhaler (SMI).

In embodiments, a breath-actuated inhaler includes an actuator housing for holding the medicament. A canister is removably received within the actuator housing. The canister contains a fluid formulated with the medicament and may be embodied as an aerosol canister. In embodiments, the fluid formulated with the medicament may be stored in a reservoir. The canister may have a generally cylindrical structure. The canister includes a metering valve for metering an amount of the medicament exiting the canister corresponding to a single spray pattern or spray plume. The canister releases a predetermined amount of the medicament through the metering valve upon actuation. The canister further includes a valve stem extending from the metering valve. At a closed bottom end of the actuator housing sits a nozzle block that includes a stem socket. The stem socket is provided for receiving the valve stem of the canister. The stem socket includes an exit orifice or actuator nozzle communicating with a mouthpiece of the inhaler. On inhalation by the patient through the mouthpiece, a pressure differential in the actuator housing causes the canister to displace relative to the valve stem. The medicament contained within the metering chamber of the canister is accordingly released in response to the patient's inspiration. During the patient's inspiration, air flows from an air inlet and through the actuator housing. The medicament released from the canister enters this air flow. Thus, during operation of the inhaler, a plume of the medicament is inhaled by the patient through the mouthpiece.

In embodiments, such as depicted in FIG. 1, the inhaler 100 is a press-and-breathe inhaler. The inhaler 100 includes an actuator housing 102 and a mouthpiece 104 defined at a lower end 106 of the actuator housing 102. Further, the actuator housing 102 receives a canister (not depicted) having a generally cylindrical structure and a metering valve. The canister releases a spray of medicament when the canister is depressed by the patient, via the metering valve. In the inhaler 100, the spray may be introduced directly into the patient's mouth, nasal area, or respiratory airways. The inhaler 100 is actuated by pressure applied by the patient's fingers, button action, or other related manual techniques.

The actuator housing 102 defines an outer surface 108 having a grip section (not depicted). The grip section allows a user to grip the inhaler 100 while using the inhaler 100. The actuator housing 102 also includes a display device (not depicted) for providing notifications to the patient. For example, the display device may notify the patient when the medicament in the canister is about to deplete. Further, the actuator housing 102 includes an air inlet (not depicted) for receiving air flow. The air inlet may be defined at an upper end 110 or the lower end 106 of the actuator housing 102.

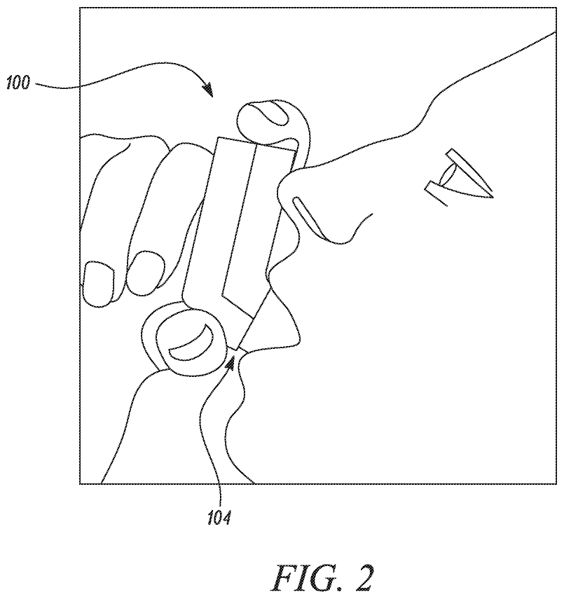

Further, the mouthpiece 104 is embodied as a generally tubular portion extending from the actuator housing 102. The mouthpiece 104 is joined to the actuator housing 102. In an example, the mouthpiece 104 is angled with respect to the actuator housing 102. The mouthpiece 104 may have a circular cross-section or a non-circular cross-section, such as an elliptical or oblong cross-section. The present disclosure is not limited by a type of non-circular shape of the cross-section of the mouthpiece 104. Further, the mouthpiece 104 has a substantially hollow structure. Referring to FIG. 2, a user or patient may put at least a part of the mouthpiece 104 into his mouth for using the inhaler 100.

On pressing of the canister by the patient, the medicament contained within the canister is released. The medicament released from the canister enters the air flow from the air inlet. Thus, during operation of the inhaler 100, a plume of the medicament produced is inhaled by the patient through the mouthpiece 104. The inhaler 100 may also include an integrated dose counter (not depicted) that may assist in indicating to the patient when the medicament in the canister is about to deplete and provide health monitoring data to health personnel. In some cases, the lips of the patient may not be sealed around the mouthpiece 104 and/or their tongue may be in a high position which may block the mouthpiece 104. Such blockage of the mouthpiece 104 due to improper positioning of the patient's mouth may lead to incomplete or ineffective delivery of the medicament to the patient.

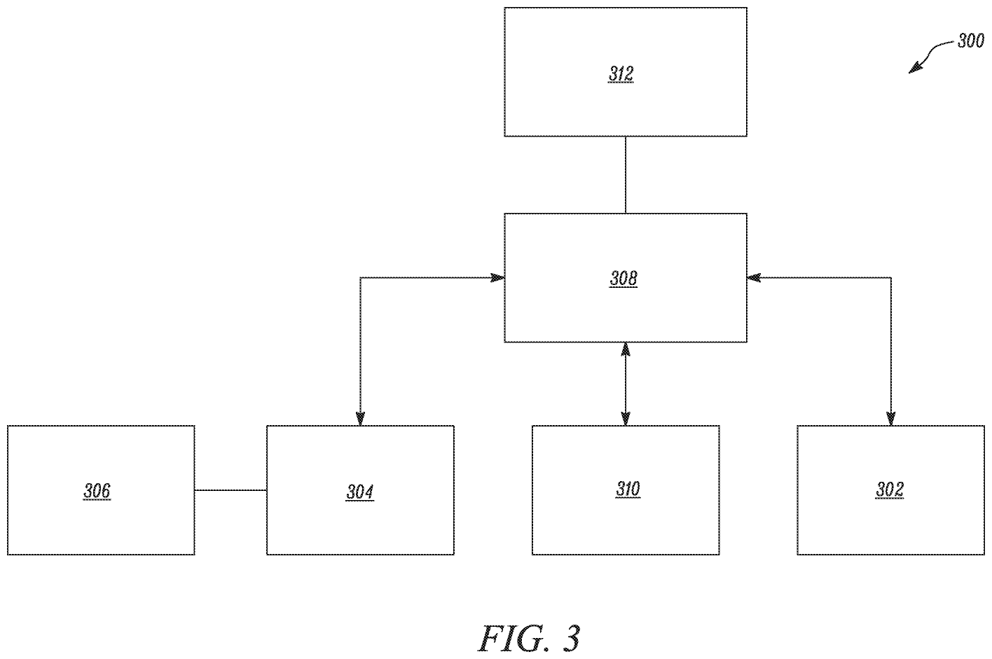

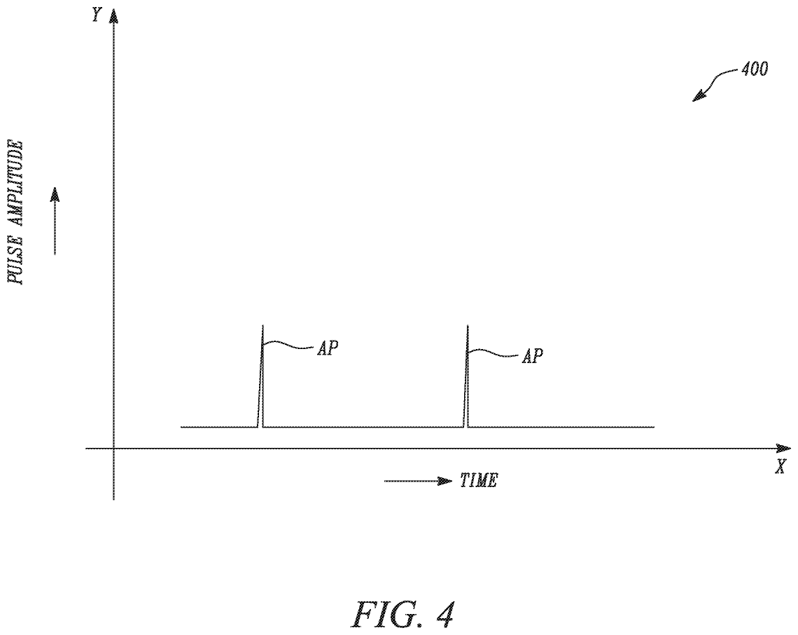

Referring to FIG. 3, the present disclosure is related to a system 300 for use with the inhaler 100. The system 300 includes a loudspeaker 302 (also depicted in FIG. 1) disposed within the actuator housing 102. The loudspeaker 302 is configured to emit an acoustic pulse “AP” during use of the inhaler 100 by the patient. In embodiments, the acoustic pulse “AP” is a Dirac pulse. In an example, the loudspeaker 302 may be embodied as a Test Tone Generator from EsserAudio.com which is connected to a small high impedance speaker inserted within the actuator housing 102 to produce the Dirac pulse. FIG. 4 illustrates exemplary acoustic pulses “AP” generated by the loudspeaker 302. The acoustic pulse “AP” is illustrated on a plot 400 with time marked on X-axis and pulse amplitude marked on Y-axis.

As depicted in FIG. 3, the system 300 further includes a microphone 304 (also depicted in FIG. 1) disposed within the actuator housing 102. It should be noted that the loudspeaker 302 and the microphone 304 may be connected to the controller 308 in a wired or wireless manner. In an example, the actuator housing 102 may include an opening provided on one side of the actuator housing 102. When the loudspeaker 302 and the microphone 304 are connected to the controller 308 in the wired manner, the opening may allow wires 314 (see FIG. 1) to pass therethrough. The wires 314 may allow wired connection of the loudspeaker 302 and the microphone 304 with the controller 308. In an example, the microphone 304 may include an omnidirectional microphone, without any limitations. The microphone 304 is configured to receive reflected sound generated in response to the acoustic pulse “AP”. The reflected sound may be embodied as a convulsion signal that is generated based on the acoustic pulse “AP” and an in-breath of the patient. In an example, a muffle 306 is disposed around the microphone 304. The muffle 306 optimizes the microphone 304 which in turn influences an ability of the microphone 304 to detect the air flow through the actuator housing 102. In some embodiments, the microphone may be a directional microphone, such as a unidirectional microphone.

Controller 308 may embody a single microprocessor or multiple microprocessors for receiving signals from components of the system 300. Numerous commercially available microprocessors may be configured to perform the functions of the controller 308. The controller 308 may further include a memory to store data, software, and algorithms therein. In some examples, the controller 308 may include a recording device and one or more processing software to process received signals. Further, the controller 308 may be configured to control the loudspeaker 302 to emit the acoustic pulse “AP” immediately prior to, during, and/or after an inhalation of the patient. The controller 308 is communicably coupled to the microphone 304. The controller 308 is also communicably coupled to the loudspeaker 302. The controller 308 is configured to analyze the reflected sound received by the microphone. In embodiments, the analyzed sound may be used to determine a shape of the oral cavity of the patient.

In embodiments, the loudspeaker and/or the microphone is disposed in the mouthpiece.

The controller 308 receives the reflected sound and processes it to determine one or more parameters of the oral cavity of the patient. Useful parameters include the shape of the oral cavity of the patient, positioning of the tongue, and positioning of the lips. More particularly, the controller 308 receives the reflected sound from the microphone 304 and generates a pattern corresponding to spectral or frequency response for the reflected sound. In an example, the controller 308 runs an algorithm, such as a Fast Fourier transform, to generate the spectral responses corresponding to the reflected sound, without any limitations. Further, the controller 308 compares the generated pattern with one or more predetermined signal patterns indicative of particular oral cavity shapes and/or tongue positions and/or positions of the lips. The predetermined signal patterns may be stored in the memory of the controller 308 or a database and may be retrieved as and when required. Further, if a signal (e.g., a peak signal) of the pattern generated based on the reflected sound differs from a signal of a predetermined signal pattern for the correct tongue position or sealing of the lips, the controller 308 generates a feedback signal for alerting the patient that the patient has not positioned his mouth correctly around the mouthpiece 104. However, if the signal (e.g., a peak signal) of the pattern generated based on the reflected sound is similar to the signal of a predetermined signal pattern for the correct tongue position or sealing of the lips, the controller 308 generates a feedback signal indicating that the patient has positioned his mouth correctly around the mouthpiece 104.

Additionally, the system 300 may also include an output device 312 that is communicably coupled to the controller 308. The output device 312 may generate a notification or alert the patient if the patient has not positioned his mouth correctly around the mouthpiece 104 (for example has not positioned his tongue correctly (e.g., blocking delivery of medicament to the airways) or has not properly sealed his lips around the mouthpiece 104). Further, when the patient has good inhalation technique, the output device 312 may provide a feedback signal indicating that the patient has positioned his mouth correctly around the mouthpiece 104. In an example, the output device may be embodied as the display device that is already present on the inhaler 100. Further, in some examples, the controller 308 may send the data corresponding to the positioning of the inhaler 100 to health care professionals. The controller may send the data by wireless communication to a remote database for storage, to an electronic database stored and downloadable from the inhaler, or to a remote communication device such as a smartphone. Further, the output device 312 may include at least one of an optical device, an audio device, and a haptic device. In some examples, the output device 312 may include a single output device or a combination of output devices that generate the feedback signal. The optical device may be a Light Emitting Diode (LED) wherein the feedback is issued based on illumination of a specific color on the LED. The audio device may be a buzzer wherein the feedback is issued based on a sound generated by the buzzer or the audio device may generate a text message. Alternatively, the optical and audio devices may include any other known output device that issues the feedback, without limiting the scope of the present disclosure. The output device 312 may be positioned on the actuator housing 102. Further, the output device 312 may also include a haptic device that provides a haptic vibration to the patient to issue the feedback. In some embodiments, the output device may provide both an audio warning and a vibrational warning.

Further, the controller 308 is configured to determine the position of the tongue of the patient based on the reflected sound. Additionally, the controller 308 is configured to determine whether the tongue of the patient is obstructing an airway of the patient based on the reflected sound.

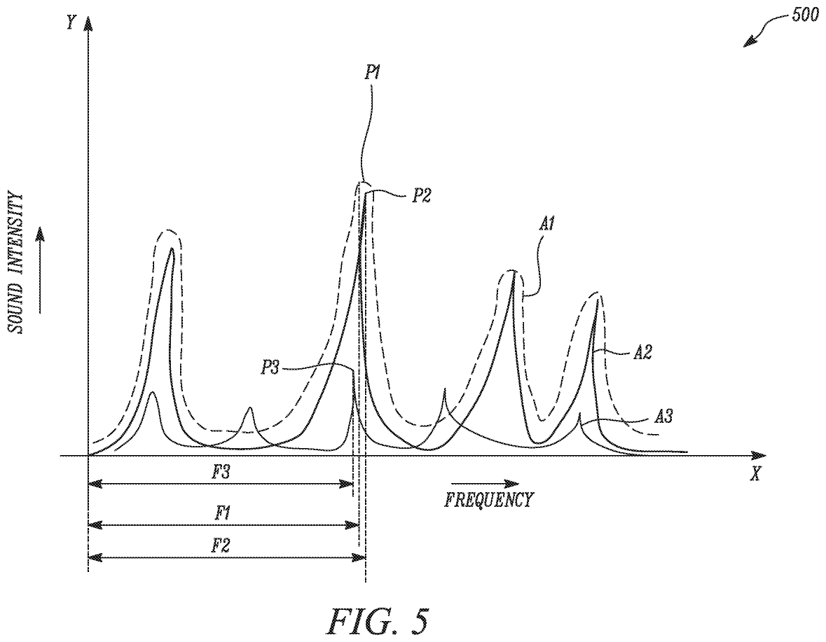

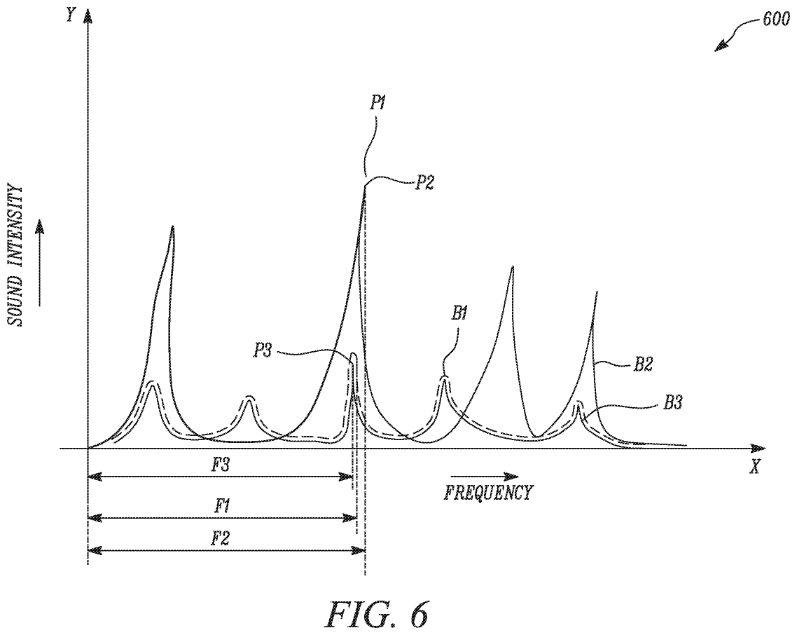

FIGS. 5 and 6 illustrate exemplary plots 500, 600, with frequency in Hz marked on X-axis and sound intensity in decibels marked on Y-axis, for tests that can be performed to determine whether a person has positioned their tongue correctly relative to the mouthpiece 104.

In one test, a person makes an “Ah” sound on an out-breath and makes a silent in-breath while keeping the shape of the mouth the same during the process. The vowel sound “Ah” is generated when the tongue is in a low position in the mouth. In a low position, the tongue is not blocking delivery of medicament dispensed by the inhaler 100 to an airway. When the tongue is in a low position, it is positioned near the bottom of the mouth and is laying generally flat rather than angled upwards towards the roof of the mouth. In the low position, the tongue is generally positioned proximal to the lower set of teeth.

In another test, a person positions their mouth around the mouthpiece 104 and makes the vowel sound “Ee” on an out-breath and makes a silent in-breath while keeping the shape of the mouth the same during the process. The sound “Ee” is generated when the tongue is in a high position in the mouth. In a high position, the tongue can block delivery of medicament dispensed by the inhaler 100 to an airway. When the tongue is in a high position, the tip of the tongue is angled upwards toward the roof of the mouth. In a high position, the tip of the tongue is positioned proximal to the upper set of teeth or roof of the mouth. In some cases, when in the high position, the tip of the tongue may be touching or almost touching the upper part of the oral cavity such as upper teeth or the roof of the mouth.

The spectral and frequency responses generated by either one or both tests can be used as a basis for determining whether correct and/or incorrect placement of the tongue has occurred during inhalation of medicament from the inhaler. The spectral and frequency responses of the tests test can be stored in the memory of the controller. The plot 500 illustrates a detected pattern “A1” which is generated by the controller 308 corresponding to the reflected sound based on a person's in-breath and the acoustic pulse “AP”. The pattern “A1” may be generated on a real time basis during use of the inhaler 100 by the person. The pattern may be used by the system 300 to provide feedback regarding correct usage of the inhaler 100. Further, the plot 500 illustrates a pattern “A2” which is generated based on previous test measurements and corresponds to the reflected sound that is generated when a person breathes in with the tongue in a high position and the mouth is in a position appropriate of forming the vowel sound “Ee”. Additionally, the plot 500 illustrates a pattern “A3” which is generated based on previous test measurements and corresponds to the reflected sound that is generated when a person breathes in with the tongue in a low position and the mouth is in a position appropriate of forming the vowel sound “Ah”. It should be noted that the patterns “A2”, “A3” are embodied as predetermined signal patterns that are generated and stored in the memory of the controller 308 based on previous measurements for different tongue positions.

As illustrated, the predetermined signal pattern for a person breathing in with the mouth in the position appropriate of forming the vowel sound “Ah” results in a peak signal “P3” produced at a frequency “F3” of the pattern “A3” that is not very high. Further, the predetermined signal pattern for a person placing his mouth around the mouthpiece 104 of the actuator housing 102 and breathing in with the mouth in the position appropriate of forming the vowel sound “Ee” results in a peak signal “P2” produced at a frequency “F2” that is greater as compared to the peak signal “P3”. In an example, the frequency “F2” may correspond to about 1000 Hz or about 1100 Hz. In some examples, additional higher frequency resonances may also be observed around frequencies that are higher than the frequency “F2”. As illustrated in FIG. 5, the controller 308 detects a signal pattern A1 with a peak signal “P1” that is similar to the peak signal “P2”. Thus, the controller 308 determines that the person's mouth was not positioned correctly around the mouthpiece 104.

The frequency “F2” may correspond to about 1000 Hz to about 1200 Hz. In some examples, additional higher frequency resonances may also be observed around frequencies that are higher than the frequency “F2”. Additionally, in some examples, there may be a significant reduction in the peak signal “P2” at a frequency that is greater than the frequency “F2”. In one example, the frequency at which there is a significant reduction in the peak signal “P2” is approximately 1500 Hz.

The test results depicted in FIG. 6 correspond to a spectral response generated based on the test performed by a person, wherein the person positions their mouth around the mouthpiece 104 and keeps the mouth in the shape of making the vowel “Ah” during in-breath and out-breath.

The plot 600 illustrates a detected pattern “B1” which is generated by the controller 308 corresponding to the reflected sound based on the person's in-breath and the acoustic pulse “AP”. The pattern “B1” may be generated on a real time basis during use of the inhaler 100 by the person. The pattern may be used by the system 300 to provide feedback regarding correct usage of the inhaler 100. Further, the plot 600 illustrates a pattern “B2” which is generated based on previous test measurements and corresponds to the reflected sound that is generated when a person breathes in with the tongue in the high position and mouth is in the position appropriate of forming the vowel sound “Ee”. Additionally, the plot 600 illustrates a pattern “B3” which is generated based on previous test measurements and corresponds to the reflected sound that is generated when a person breathes in with the tongue in the low position and the mouth is in the position appropriate of forming the vowel sound “Ah”. It should be noted that the patterns “B2” and “B3” are embodied as predetermined signal patterns that are generated and stored in the memory of the controller 308 based on previous measurements for different tongue positions.

In the illustrated example, the controller 308 detects that a peak signal “P 1” is similar to the peak signal “P3”. Thus, the controller 308 determines that the person has positioned their mouth correctly around the mouthpiece 104.

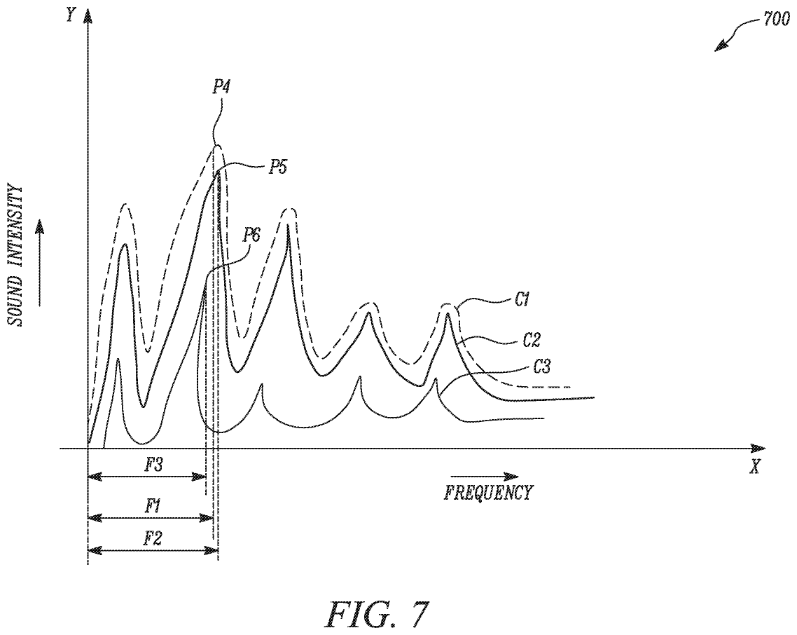

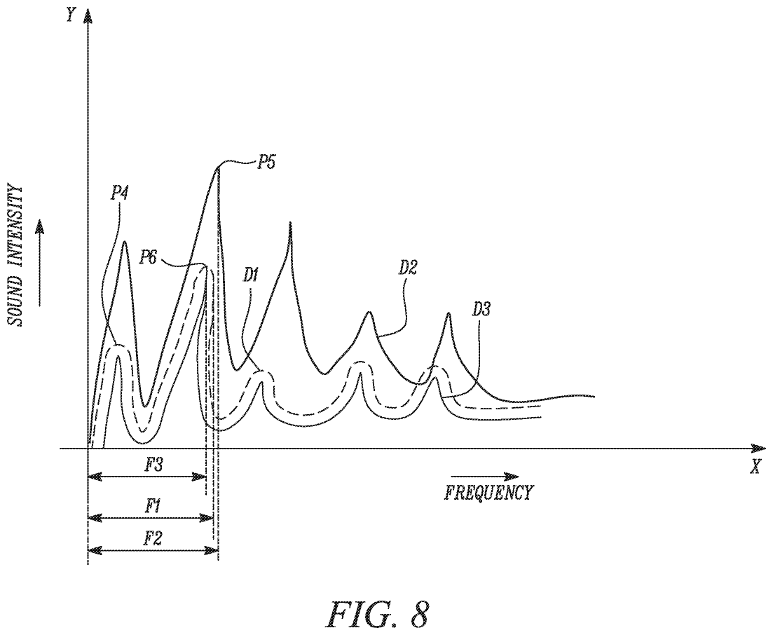

In embodiments, the controller 308 may be configured to determine whether the lips of a person are sealed around the mouthpiece 104 of the actuator housing 102 based on the reflected sound. FIGS. 7 and 8 illustrate exemplary plots 700, 800 with frequency in Hz marked on X-axis and sound intensity in decibels marked on Y-axis for tests that were performed to determine whether a person has sealed their lips correctly around the mouthpiece 104. Referring to FIG. 7, the plot 700 illustrates spectral or frequency response generated based on the test performed by a person. In this test, the person positions their mouth around the mouthpiece 104 such that their lips seal around the mouthpiece 104.

The plot 700 illustrates a detected pattern “C1” by the controller 308 which is generated corresponding to the reflected sound based on a person's in-breath and the acoustic pulse “AP”. The pattern “C1” may be generated on a real time basis during use of the inhaler 100 by a person. The pattern may be used by the system 300 to provide feedback regarding correct usage of the inhaler 100. Further, the plot 700 illustrates a pattern “C2” which is generated based on previous measurements and corresponds to the reflected sound that is generated when a person seals their lips around the mouthpiece 104. Additionally, the plot 700 illustrates a pattern “C3” which is generated based on previous measurements and corresponds to the reflected sound that is generated when a person does not seal their lips around the mouthpiece 104. It should be noted that the patterns “C2”, “C3” are embodied as predetermined signal patterns that are generated and stored in the memory of the controller 308 based on previous measurements corresponding to sealing of the lips.

As illustrated, the predetermined pattern for a person sealing their lips around the mouthpiece 104 results in a peak signal “P5” produced at a frequency “F2” of the pattern “C2” that is high. Further the predetermined pattern for a person not sealing their lips around the mouthpiece 104 results in a peak signal “P6” produced at a frequency “F3” that is lesser as compared to the peak signal “P5”. In the illustrated example, the controller 308 detects that a peak signal “P4” is similar to the peak signal “P5”. Thus, the controller 308 determines that the person's lips were sealed around the mouthpiece 104.

Referring now to FIG. 8, the plot 800 illustrates spectral or frequency response generated based on the test performed by a person. In this test, the person positions their mouth around the mouthpiece 104 such that their lips do not seal around the mouthpiece 104. The plot 800 illustrates a detected pattern “D1” which is generated by the controller 308 corresponding to the reflected sound based on the person's in-breath and the acoustic pulse “AP”. The pattern “D1” may be generated on a real time basis during use of the inhaler 100 by the person. The pattern may be used by the system 300 to provide feedback regarding correct usage of the inhaler 100. Further, the plot 800 illustrates a pattern “D2” which is generated based on previous measurements and corresponds to the reflected sound that is generated when a person seals their lips around the mouthpiece 104. Additionally, the plot 800 illustrates a pattern “D3” which is generated based on previous measurements and corresponds to the reflected sound that is generated when a person does not seal their lips around the mouthpiece 104. It should be noted that the patterns “D2”, “D3” are embodied as predetermined signal patterns that are generated and stored in the memory of the controller 308 based on previous measurements corresponding to sealing of the lips.

As illustrated, when a person seals their lips around the mouthpiece 104, a peak signal “P5” produced at a frequency “F2” of the pattern “D2” is high. Further, had the person not sealed their lips around the mouthpiece 104, then a peak signal “P6” would have resulted at a frequency “F3” that is lesser as compared to the peak signal “P5”. In the illustrated example, the controller 308 detects that a peak signal “P4” is similar to the peak signal “P6”. Thus, the controller 308 determines that the person has not sealed their lips around the mouthpiece 104. =

Further, in an example, the controller 308 runs an algorithm, such as a Fast Fourier transform, to generate the spectral responses corresponding to the plots 500, 600, 700, 800, without any limitations.

The embodiments described in FIGS. 5-8 are exemplary. Alternative breathing patterns and/or mouth shapes can generate other patterns that may be useful for distinguishing between proper and improper inhaler technique. For example, a patient may take a deep breath with the tongue in the low position, a deep breath with the tongue in the high position, breathing through nose, heavy breathing, gentle breathing, etc.

It should also be understood, that the controller may be programmed to analyze multiple peaks in a reflected sound plot, one or more subsections of a reflected sound plot, or even an entire reflected sound plot. The controller may be programmed to analyze for different sound patterns based on sound frequency and/or sound intensity of a reflected sound plot. The controller may analyze the reflected sound based on sound frequency, sound intensity or combinations thereof.

The system 300 may also include a passive acoustic device 310 disposed within the actuator housing 102 and communicably coupled to the controller 308. The passive acoustic device 310 is configured to monitor at least one of an inhalation flowrate, a total volume of inhalation, and a timing of actuation of the inhaler 100 within an inhalation cycle. More particularly, the system 300 can be used in conjunction with the passive acoustic device 310 by operating the loudspeaker 302 in a pulsed mode so that patient attributes, such as the inhalation flowrate, the total volume of inhalation, and the timing of actuation within the inhalation cycle can be monitored. The passive acoustic device 310 may include, for example, an acoustic sensor.

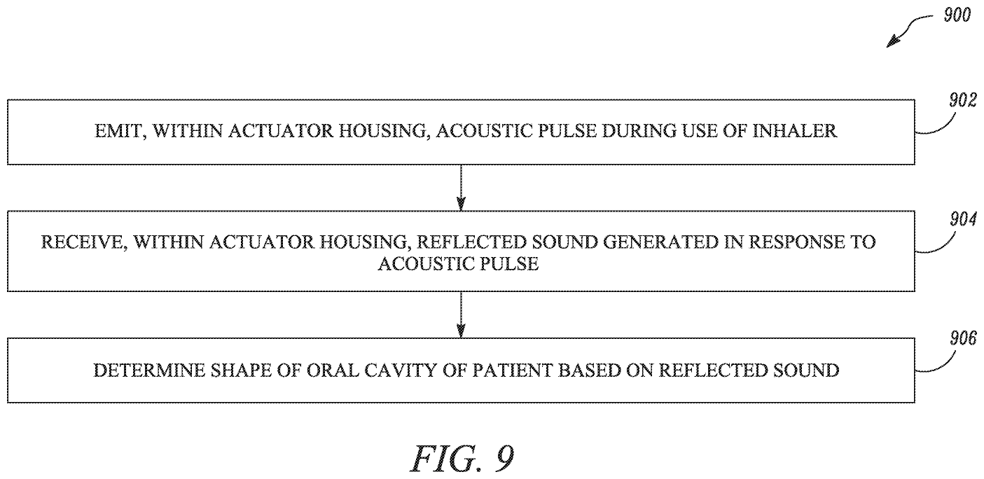

FIG. 9 is a flowchart for a method 900 of monitoring use of the inhaler 100 by the patient. The inhaler 100 includes the actuator housing 102. At step 902, the acoustic pulse “AP” is emitted within the actuator housing 102 during use of the inhaler 100. The acoustic pulse “AP” is generated by the loudspeaker 302. The acoustic pulse “AP” may be a Dirac pulse. Further, the acoustic pulse “AP” is generated immediately prior to, during, and/or after the inhalation of the patient. At step 904, the reflected sound generated in response to the acoustic pulse “AP” is received within the actuator housing 102. The reflected sound is received by the microphone 304.

At step 906, a parameter of the patient's oral cavity is determined based on the reflected sound. As depicted in FIG. 9, the determined parameter is the shape of the oral cavity. Other parameters that might be determined include the positioning of the tongue and positioning of the lips. In embodiments, the method 900 also includes a step of determining whether the tongue is obstructing the airway of the patient based on the reflected sound. In embodiments, the method 900 includes a step of determining whether the lips of the patient are sealed around the mouthpiece 104 of the actuator housing 102 based on the reflected sound. In embodiments, the method 900 may also include a step of monitoring at least one of the inhalation flowrate, the total volume of inhalation, and the timing of actuation of the inhaler within the inhalation cycle

It should be noted that a position of the microphone 304 and the loudspeaker 302 mentioned above may vary based on desired acoustic performance. Also, a design of the actuator housing 102 may be optimized to maximize the acoustic performance. Further, the system 300 described above may form a part of an electronic inhaler such as described in International Patent Application Publication WO 2017/112400, “Medicinal Inhalers”. The systems, methods, and devices of the present disclosure can be used for various inhalation devices, such as, by way of example, pMDI, DPI, and SMI. The systems, methods, and devices of the present disclosure may also help in providing valuable feedback to both the patients and the health care professionals thereby improving adherence monitoring. Additionally, the system 300 is easy to implement and is cost effective. Further, the system 300 can be applied to inhalers of press-and-breathe type, breath-actuated types, etc., without limiting the scope of the present disclosure.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments depicted and described without departing from the scope of the present disclosure. This application is intended to cover any adaptations or variations of the specific embodiments discussed herein. Therefore, it is intended that this disclosure be limited only by the claims and the equivalents thereof.

1-20. (canceled)

21. A system for use with an inhaler, the inhaler having an actuator housing having a mouthpiece, the system comprising:
a loudspeaker disposed within the actuator housing, wherein the loudspeaker is configured to emit an acoustic pulse during use of the inhaler by a patient;
a microphone disposed within the actuator housing, wherein the microphone is configured to receive reflected sound generated in response to the acoustic pulse; and
a controller communicably coupled to the microphone, the controller configured to analyze the reflected sound received by the microphone and determine based on the reflected sound at least one of a position of the tongue of a patient or whether lips of the patient are sealed around the mouthpiece of the actuator housing.

22. The system of claim 21, wherein the controller is configured to determine the shape of the oral cavity.

23. The system of claim 21, wherein the controller is further configured to determine whether the tongue of the patient is obstructing an airway of the patient based on the reflected sound.

24. The system of claim 21, further comprising a muffle disposed around the microphone.

25. The system of claim 21, wherein the controller is further configured to control the loudspeaker to emit the acoustic pulse immediately prior to, during, and/or after an inhalation of the patient.

26. The system of claim 21, further comprising a passive acoustic device disposed within the actuator housing and communicably coupled to the controller, the passive acoustic device being configured to monitor at least one of an inhalation flowrate, a total volume of inhalation, and a timing of actuation of the inhaler within an inhalation cycle.

27. A method of monitoring use of an inhaler by a patient, the inhaler having an actuator housing, the method comprising:
emitting, within the actuator housing, an acoustic pulse during use of the inhaler;
receiving, within the actuator housing, reflected sound generated in response to the acoustic pulse; and
determining a parameter of the patient's oral cavity based on the reflected sound, wherein determining the parameter of the oral cavity further comprises determining based on the reflected sound at least one of a position of the tongue of a patient or whether lips of the patient are sealed around a mouthpiece of the actuator housing.

28. The method of claim 27, wherein determining the parameter of the oral cavity further comprises determining the shape of the oral cavity.

29. The method of claim 27, further comprising determining whether the tongue is obstructing an airway of the patient based on the reflected sound.

30. The method of claim 27, further comprising emitting the acoustic pulse immediately prior to, during, and/or after inhalation.

31. The method of claim 27, further comprising the monitoring at least one of an inhalation flowrate, a total volume of inhalation, and a timing of actuation of the inhaler within an inhalation cycle.

32. An inhaler for delivering a medicament to a patient, the inhaler comprising:
an actuator housing comprising a mouthpiece:
a loudspeaker disposed within the actuator housing, wherein the loudspeaker is configured to emit an acoustic pulse during use of the inhaler by the patient;
a microphone disposed within the actuator housing, wherein the microphone is configured to receive reflected sound generated in response to the acoustic pulse; and
a controller communicably coupled to the microphone, the controller configured to analyze the reflected sound received by the microphone and determine based on the reflected sound at least one of a position of the tongue of a patient or whether lips of the patient are sealed around the mouthpiece of the actuator housing based on the reflected sound.

33. The inhaler of claim 32, wherein the controller is further configured to determine whether the tongue of the patient is obstructing an airway of the patient based on the reflected sound.

34. The inhaler of claim 32, further comprising a passive acoustic device disposed within the actuator housing and communicably coupled to the controller, the passive acoustic device being configured to monitor at least one of an inhalation flowrate, a total volume of inhalation, and a timing of actuation of the inhaler within an inhalation cycle.
